(19) United States
(12) Patent Application Publication
(10) Pub. No.: US 2020/0281928 A1
(43) Pub. Date: Sep. 10, 2020

(54) VITAMIN B1 IN HIGH DOSES FOR USE IN THE MEDICAL TREATMENT OF MOTOR SYMPTOMS OF SOME SPORADIC NEURODEGENERATIVE DISEASES, OF GENETIC ORIGIN, AND OF CLUSTER HEADACHE AND OF MIGRAINE HEADACHE

(71) Applicants: Antonio COSTANTINI, Viterbo (IT); Marco COLANGELI, Viterbo (IT); Maria PALA, Viterbo (IT); Robert FANCELLU, Genova (IT)

(72) Inventor: Antonio COSTANTINI, Viterbo (IT)

(21) Appl. No.: 16/618,667

(22) PCT Filed: May 11, 2018
(86) PCT No.: PCT/IB2018/053291

§ 371 Date: Dec. 2, 2019

(30) Foreign Application Priority Data
May 31, 2017 (IT) 102017000059814

(51) Int. Cl. A61K 31/51

(52) U.S. Cl. CPC A61K 31/51; A61K 9/0053

(57) ABSTRACT

The present invention relates to the use of vitamin B1 and to pharmaceutical compositions including it, in the treatment of motor symptoms of some sporadic neurodegenerative diseases (that is not of genetic origin), of genetic origin and of the primary headaches (cluster headache and migraine headache). In particular the present invention relates to vitamin B1 for use in the treatment of motor symptoms of one of the following pathologies: Essential Tremor (also called induced tremor), Dystonia 1 (DYT1), Huntington's Chorea, Sporadic spinocerebellar ataxia, Atypical Parkinsonisms (multisystemic atrophy of Parkinson type and Cerebellar type, progressive supranuclear paralysis, Cortico-basal degeneration); and headache episodes of Cluster headache and Migraine headache.

The present invention relates to the use of vitamin B1 and to pharmaceutical compositions including it, in the treatment of motor symptoms of some sporadic neurodegenerative diseases (that is not of genetic origin), of genetic origin and in the treatment of primary headaches, cluster headache and migraine headache.

In particular the present invention relates to vitamin B1 for use in the treatment of one of the following pathologies:

Diseases characterized by a motion disorder:

Essential Tremor (also called induced tremor), Dystonia 1 (DYT1), Huntington's Chorea, Sporadic spinocerebellar ataxia, Atypical Parkinsonisms (multisystemic atrophy of Parkinson type and Cerebellar type, progressive supranuclear paralysis, Cortico-basal degeneration);

Diseases characterized by serious pain to the head (with episodic or continuous attacks) that is Cluster headache and Migraine headache.

What all these pathologies with symptoms very different (motor disorders in a group, pain in headaches) and apparently very far therebetween have in common is the fact that the primary cause, whatever it is, could determine a dysfunction of the intracellular thiamine-dependent mechanisms and then a mitochondrial dysfunction with consequent involvement of the energy metabolism in determined nerve centres. This deficit is localized, focal, and accounts for the particular symptoms and course of each single mentioned pathology. The localization of the dysfunction at the level of the motor systems causes tremor, incoordination, dystonia, bradykinesia, lack of motion, posture instability, plastic hypertonia, involuntary motions.

The localization at the hypothalamus level, in the vegetative centres, in the nociceptive routes causes the symptoms of cluster headache and of migraine headache (head pain with particular characteristics and localization).

The involvement of the thiamine-dependent mechanisms, that is a deficiency in intracellular thiamine in the above-mentioned diseases, explains the effectiveness of high doses of vitamin B1 orally or intramuscularly in treating such pathologies.

### STATE OF ART

Neurodegenerative diseases are diseases wherein a progressive and inexplicable suffering of groups of neurons generally belonging to functional systems specific for each single disease is noted. From the anatomopathological point of view first of all atrophy of neurons and then their rarefaction, due to the death of the same, is noted. Usually a functional and anatomical system relating the motion is affected most seriously, therefore the diseases at issue are called motion disorders. In the listed pathologies, that is Essential Tremor (also called intention tremor), Dystonia 1 (DYT1), Huntington's Chorea, Sporadic spinocerebellar ataxia, Atypical Parkinsonisms (multisystemic atrophy of Parkinson type and Cerebellar type, progressive supranuclear paralysis, Cortico-basal degeneration), the most affected centres are the nuclei of the base, the cerebellum, the brainstem, often the limbic system and the cerebral cortex are affected.

Primary headaches (cluster headache and migraine headache) are characterized by the pain localized in one or more areas of the skull. In this case it is a disorder involving the pain routes, the nociceptive centres of mesencephalon and hypothalamus (in these last pathologies one is not sure about the presence of neurodegenerative facts in the affected centres).

In the current state of art for the above-mentioned motion disorders there is no effective medical therapy, therefore they are considered incurable diseases which progressively lead to disability and death.

The primary headaches (cluster headache and migraine headache) are characterized by the pain localized in one or more areas of the skull. In the current state of art there is no valid medical therapy. There is a symptomatic therapy, not always effective and not without serious side effects.

Then, the need for providing new compositions for the treatment of these pathologies is much felt.

### SUMMARY OF THE INVENTION

The present invention is based upon the fact of noting that by administering vitamin B1 (thiamine) at high doses, orally or intramuscularly, to patients affected by subject neurodegenerative pathologies, described in details hereinafter, the motor symptoms are reduced in a clearly appreciable way (sometimes until almost complete regression). The treatment, continued over time, further slows down or stops the disease progression.

The administration of vitamin B1 at high doses orally or intramuscularly, improves clearly or makes the episodes of headache pain in the cluster headache and migraine headache to disappear completely.

The high doses can be both oral and intramuscular according to the equivalence dosage described hereinafter in this document.

The observed therapeutic effect is still more surprising considering that all treated patients had values of vitamin B1 in plasma thoroughly normal. These values then suggested that a vitamin B1-based treatment was not necessary and that it would not have determined any therapeutic effect.

Even if one does not want to link the present invention to a specific mechanism, one can assume that the observed therapeutic effect is the following. The normal concentration of vitamin in plasma, associated to the presence of symptoms of deficiency of the same, indicates to stop using thiamine at intracellular level. In other words, the vitamin goes with difficulty from blood to tissues, or it finds obstacles in the passage inside the cells, or still there is something (as an enzymatic structural alteration may be) preventing from using it at the level of mitochondria of the various organs and apparatuses in particular of encephalon.

In other words, the pathogenesis of the symptoms could be attributed to an intracellular deficiency of thiamine. The administered high doses increase the concentration of vitamin B1 in the blood at a level so that the passive transmembrane transportation towards the interior of the cells increases the concentration of vitamin B1 inside cytoplasm and mitochondria until restoring the thiamine-dependant metabolic processes which are fundamental to produce energy from glucose. The produced energy is the one which guarantees the functionality of the cells' metabolic processes by keeping them capable of carrying out their function and of living.

The same happens in the primary headaches. The malfunction of the cerebral centres triggering the pain can be attributed to an intracellular thiamine deficiency. In fact, high doses of vitamin B1 are capable of abolishing the headache attacks.

Therefore the present invention relates to vitamin B1 for use in the treatment of the symptoms of a pathology selected in the group of:

1) Essential Tremor, Dystonia type DYT1, Huntington's Chorea, Sporadic spinocerebellar ataxia, and Atypical Parkinsonisms, in particular multisystemic atrophy of Parkinson type and Cerebellar type, progressive supranuclear paralysis, Cortico-basal degeneration.

2) Cluster headache, Migraine headache

The invention further relates to pharmaceutical compositions including vitamin B1 and one or more pharmacologically acceptable excipients for use in the treatment of the symptoms of a pathology selected in the group of:

1) Essential Tremor, Dystonia type DYT1, Huntington's Chorea, Sporadic spinocerebellar ataxia, and Atypical Parkinsonisms, in particular multisystemic atrophy of Parkinson type and Cerebellar type, progressive supranuclear paralysis, Cortico-basal degeneration.

2) Cluster headache, Migraine headache

The advantages, features and use modes of the present invention will result evident from the following detailed description of some embodiments, shown by way of example and not for limitative purposes.

### DETAILED DESCRIPTION OF THE INVENTION

According to the present invention vitamin B1 is administered to the patients affected by some diseases of neurodegenerative type of genetic or unknown origin (sporadic degenerative diseases) or affected by cluster headache or by headache of migraine type.

In the present description under the term neurodegenerative pathologies a group of diseases is meant wherein a progressive, inexplicable atrophy and subsequent death of determined neuronal groups takes place. The affected patient has progressively motor deficits which, in more or less periods of time, lead him/her to disability and in some types of pathology to death. In particular vitamin B1 could be used in treating motor symptoms of these pathologies:

Essential Tremor (also called intention tremor), Dystonia type DYT1, Huntington's Chorea, Sporadic spinocerebellar ataxia, Atypical Parkinsonisms (multisystemic atrophy of Parkinson type and Cerebellar type, progressive supranuclear paralysis, Cortico-basal degeneration) as far as exclusively the motor symptoms are concerned.

The same molecule appears very effective in treating Cluster headache and Migraine headache.

No one knows if at the base of the primary headaches (as well as in the Essential Tremor) there is a morphologically detectable neurodegenerative process. Surely in the intention tremor the cerebellar circuits appear to be involved and in the primary headaches (cluster headache and migraine headache) the hypothalamus which adjusts the vegetative system and the circadian rhythms appears to be involved. The triggering mechanisms, however, are little known. Vitamin B1 could be used even for treating cluster headache and migraine headache.

Vitamin B1 can be administered depending upon age and general conditions of the patient, nature and seriousness of the pathology or disorder and the route and type of administration. The dosage, then, will have to take into account the particular condition to be treated, seriousness of the condition to be treated, age, weight and general physical conditions of the particular patient. Moreover, it is clear that the effective quantities, if required, can be decreased or increased according to the responses of the treated patient and/or according to the physician evaluation.

However, the clinical experiments shown in the examples highlighted that the mitigation until even the complete regression of the symptoms is obtained with a dosage of vitamin B1 ranging between 300 and 8000 mg a day orally, preferably with a dosage between 2000 and 8000 mg, or with an intramuscular administration of about 25-100 mg per week and until a maximum of a 100-mg intramuscular vial a day.

The lowest doses were used in treating headaches.

In some patients the treatment by intramuscular route was alternated to the oral one and vice versa for some periods of time.

Thus, we succeeded in defining the parental equivalent and oral doses suitable to obtain an improvement or to keep an obtained result. In order to have the therapeutic effects of a determined intramuscular dose it is necessary to administer to the patient an oral dose even up to about 100, preferably 140 times higher and vice versa.

Based upon our clinical experience on the pathologies treated in this document, the equivalent intramuscular or oral dose produces the same biological effects. In particular 100 mg of vitamin B1 administered once a week, are equal to the ingestion of 14 grams orally, which we make to ingest by dividing the total dose into 7 single administrations (the patient ingests two grams orally every day, altogether in the morning, for each day of the week), in one single dose, of 2 g of vitamin B1 orally each day for 7 days. A smaller dose, for example 25 mg intramuscular dose of thiamine once a week, is equal to the ingestion of 500 mg of thiamine once a day for 7 days.

The present invention further relates to pharmaceutical compositions including vitamin B1 and one or more pharmacologically acceptable excipients for use in the treatment of one of the following pathologies: Essential Tremor (also called intention tremor), Dystonia type DYT1, Huntington's Chorea, Sporadic spinocerebellar ataxia, Atypical Parkinsonisms (multisystemic atrophy of Parkinson type and Cerebellar type, progressive supranuclear paralysis, Cortico-basal degeneration), Cluster headache (also called di Horton headache) and Migraine headache.

Typically said compositions can include an amount of vitamin B1 ranging between 100 mg and 500 mg per dosage unit. Preferably the dosage unit for oral administration ranges between 250 and 500 mg, for intramuscular administration between 25 and 150 mg, preferably 100 mg.

Under the term dosage unit in the present description the unit formulation for one single administration, for example a tablet, capsule, etc., is meant.

Under dosage unit in the present description the amount of active principle for one single administration is meant.

The pharmaceutical compositions according to the present invention can be formulated in a variety of ways depending upon the selected administration route. According to a specific embodiment of the invention, the pharmaceutical composition is suitable to the oral administration of solid forms and it can include forms such as capsules, tablets, pills, powders and granules. In these solid forms vitamin B1 can be mixed with one or more pharmaceutically acceptable inert excipients. Such excipients can be selected among those usually known in the state of art and include, but they are not limited thereto: a) carrier, such as sodium citrate and calcium phosphate, b) filler such as starch, lactose, microcrystalline cellulose, sucrose, glucose, mannitol and colloidal silica, c) humectants, such as glycerol, d) disintegrants, such as alginates, calcium carbonate, starches, starch derivatives, cellulose and polyvinylpyrrolidone derivatives, silicates and sodium carbonate e) binding agents such as carboxymethylcellulose, alginates, gelatin, polyvinylpyrrolidone, sucrose, cellulose polymer derivatives, starch derivatives f) retarding agents such as paraffin, cellulose polymers, esters of fatty acids g) absorption accelerators, such as quaternary ammonium compounds, h) wetting and surface active agents such as ethyl alcohol and glycerol monostearate, i) adsorbents, such as benthic clays and kaolin, k) lubricants such as talcum, calcium stearate, magnesium stearate, glycol polyethylene, sodium laureth sulfate, sodium stearyl fumarate j) glidants such as talcum, colloidal silica.

The forms of solid dosage, such as tablets, capsules, pills and granules, can be coated with enterica, gastric coatings or other type well known in the state of art. They can include opacifiers and can be of the type to allow the release of active ingredients only or preferably in a certain tract of intestine, in case, in a delayed way. Substances which can allow such delayed use include, but are not limited thereto, polymers and waxes.

Liquid forms suitable to an oral administration are emulsions, solutions, prepared or extemporaneous suspensions, syrups and elixir. Excipients suitable to the formulations according to the present invention in liquid form for oral use include, but are not limited thereto, diluents commonly used in the art, such as water or other solvents, solubilizing and emulsifying agents selected among ethyl alcohol, polyalcohols, propylene glycol, glycerol, polyethylenglycol and sorbitan esters. These formulations can even include sweeteners and flavouring agents selected among those well known in the state of art. Compositions suitable for pharmaceutical acceptable parenteral (preferably intramuscular) injections can include sterile aqueous solutions, dispersions, suspensions or emulsions or sterile powders for reconstitution in injectable solutions or dispersions; examples of excipients suitable thereto include, but they are not limited thereto, aqueous or not aqueous carriers, diluents, solvents or vehicles selected among: water, ethanol, polyols (propylene or polyethylene glycol, glycerol and the like), polyalcohols, isopropyl alcohol, ethyl acetate, benzyl alcohol, benzyl benzoate, propylene glycol, 1,3-butylene glycol, dimethylformammide, vegetable oils (in particular olive, cotton, peanut, maize, wheat germ, olive, castor, sesame oil), organic esters such as ethyl oleate or the like.

These compositions can even include preservatives of the antibacterial or antifungal type, selected, but not exclusively, among: parabenzoate, chlorobutanol, phenol, ascorbic acid and the like. The fact of including an isotonic agent, for example a sugar, sodium chloride or the like, can be useful too. Moreover, pharmaceutical forms with delayed absorption with agents such as, for example but not exclusively, monostearate aluminium and gelatin can be obtained.

Slow-release formulations can also be prepared by means of the techniques and products well known to the state of art. The present invention further relates to the treatment methods according to the herein-described administrations.

### EXAMPLES

### Experiments

Clinical experiments showing the activity of vitamin B1 according to the invention is shown hereinafter.

Essential Tremor (Also Called Intention Tremor)

Two patients were studied affected by such pathology, a man and a woman. The intention tremor, differently from tremor in Parkinson disease appears and increases with muscular activity. The diagnosis was made by neurologists expert in the motion disorders based upon the clinical, neuroradiological and electrophysiological data.

Before starting the treatment, both patients were evaluated with a validated scale called TETRAS (TRG ESSENTIAL TREMOR RATING ASSESSMENT SCALE). They were re-evaluated with the same scale after two months of treatment with two 100-mg/ml vials of thiamine (vitamin B1) intramuscularly performed on Monday and on Thursday morning with the same scale.

In turn, this scale is constituted by two sub-scales. The first one, Activities of Daily Living Subscale (ADL Subscale) measures the impact the tremor has in the activities of daily life and it has a score ranging from 0 (no impact) to 48 (maximum impact).

The second one, Performance Subscale measures the width and the extension of tremor on the body and it has a score ranging from 0 (no tremor) to 64 (tremor with wide shocks and widespread).

Patients:

N 1) male, 72 years old, for about three years tremor of head and hands during the voluntary motions mainly on the right and it is gradually and progressively worsening.

ADL Subscale in June 2014 scores 16; after two months of treatment scores 4 with an improvement of 75%.

Performance subscale June 2014 scores 19.5, after two months of treatment scores 5 with an improvement of 74.4%.

N 2) female, 75 years old, for about 4 years tremor of the head and of hands mainly on the right and gradually and progressively worsening.

ADL Subscale in June 2014 scores 23; after two months of treatment scores 8 with an improvement of 65.3%.

Performance subscale June 2014 scores 22, after two months of treatment scores 7 with an improvement of 68.2%.

The two patients have never suspended the treatment. During this period of time they have also ingested for some months the equivalent dose orally (4 grams each day of vitamin B1) by keeping the same results of the vials. Up to now they have had no any side effect nor any alteration in the common laboratory examinations. The treatment has demonstrated to be effective in the long term.

Dystonia 1 (or DYT1 or Dystonia Generalized with Early Onset)

The studied patients were two, a female and a male, mother and son. The disease is genetic and has a transmission of dominant type. The diagnosis was made in primary neurological centres based upon anamnestic, clinical and laboratory data. At time of first examination, the mother had generalized dystonic symptoms, the son localized at the upper and lower right arm. In both of them the symptoms started when they were 10 years old.

The molecular analysis of gene dYT1 showed in both patients the presence of heterozygosis in the deletion of a GAG (c.907_909delGAG, ensemb1 Gene ID ENSG00000136827) codon in exon 5 of gene DYT1.

They were evaluated with Fahn-Mardsen scale of dystonia (Fahn-Mardsen Rating Scale). This scale consists of two sub-scales: 1) motion scale 2) disability scale. The first one ranges from 0 (no motion disorder) to 120 (maximum intensity and extension of the motion dystonic disorder). The second one ranges from 0 (no disability) to 30 (maximum disability)

Patients:

P1. Female, 48 years old, mother of patient 2.

Motion scale before treatment 66 scores; disability scale 14 scores.

Motion scale after two months of treatment 31 scores; disability scale 9 scores.

The improvement was by 53.0% for the motion scale and by 35.8% for disability scale.

P2. Male, 29 years old.

Motion scale before treatment 4 scores; disability scale 4 scores.

Motion scale after two months of treatment 2 scores; disability scale 2 scores.

The improvement was by 50.0% for the motion scale and by 50.0% for the disability scale. They are currently under treatment by keeping the obtained results.

Huntington's Chorea

We evaluated and treated one single female patient. The disease is genetic and it has dominant transmission. The diagnosis was made in primary neurological centres based upon anamnestic, clinical and laboratory data. The molecular analysis showed genotype heterozygote for the mutation by expansion (CAG) of Huntingtina gene. The first symptoms appeared about six years ago.

The patient, 56 years ago, was evaluated with Unified Huntington's Disease Rating Scale (UHDRS). The scale consists of 31 items and ranges from 0 scores (absence of choreic motions) to 124 scores (maximum intensity and extension on the body of the involuntary motions of choreic type).

The first evaluation, before treatment was performed on Jun. 7, 2016, the second one, after two months of treatment with two vials intramuscularly 100 mg/ml per week was performed on Sep. 9, 2016.

UHDRS before treatment 62 scores, after two months of treatment 45 scores. The improvement was by 27.5%. Such improvement has remained stable up to now by continuing the same treatment.

Sporadic Spinocerebellar Ataxia

We treated two patients with two vials of thiamine 100 mg/ml twice a week. The diagnosis was made by exclusion, by excluding the genetic forms of spino cerebellar ataxia and the tumour secondary forms.

The patients were evaluated with SARA (Scale for Assessment and Rating of Ataxia), scale validated for all disorders of cerebellar type. The score of this scale ranges from 0 (absence of symptoms) to 40 (maximum deficit). The treatment has lasted for more than six months and the patients were evaluated before starting the treatment and after two months from the treatment beginning. They are still under treatment by keeping the obtained results.

The diagnoses were made by Italian primary neurological centres based upon the anamnestic, clinical, evolutionary, neuroradiological and neurophysiological and molecular chemistry data.

Patients:

N1. Man, age 77 years old, first symptoms appeared sixteen years ago with equilibrium disorders. The therapy started end November 2012. The first check after therapy was made end January 2013.

SARA scale before starting the therapy 9 scores.

SARA scale after two months of therapy 6 scores.

Improvement of motor symptoms by 33.4%

N2 Man, age 47 years old, first symptoms appeared 11 years ago.

SARA scale before starting the therapy 6 scores.

SARA scale after two months of therapy 3 scores.

Improvement of motor symptoms by 50.0%

The two patients are currently following the same treatment without therapy effectiveness decrease.

Atypical Parkinsonisms (multisystemic atrophy of Parkinson type and Cerebellar type, progressive supranuclear paralysis, Cortico-basal degeneration).

Under atypical Parkinsonisms or parkinsonisms plus a group of neurodegenerative diseases is meant which have in common some characteristics with Parkinson disease, but they are characterized by the presence of additional neurological symptoms and a different evolution. The atypical Parkinsonisms have a quick evolution and a more severe prognosis with respect to the Parkinson disease. They do to not respond to dopaminergic therapy as idiopathic Parkinson disease.

Multisystemic Atrophy (MSA) Type Parkinson (Type P)

We treated four patients affected by such pathology, two women and two men with five vials intramuscularly per week of vitamin B1. One vial included 100 mg/ml of thiamine. The injection was performed each morning for the first five days of the week. The dose was determined empirically based upon the disease progression rapidity.

Moreover, low doses of all other vitamins of group b, including folic acid, 150 mg of magnesium citrate and 150 mg of magnesium malate, were associated to the treatment.

The patients were evaluated with UPDRS (Unified Parkinson's Disease Rating Scale), scale validated for all Parkinson forms. Herein the scores of the third portion (portion III) of this scale are shown, illustrating the motor problems found by the examiner. The third portion ranges from 0 scores (no deficit) to 56 scores (maximum deficit), the treatment has lasted for more than six months and the patients were evaluated before starting the treatment and after two months from the treatment beginning. They are currently under treatment by keeping the obtained results.

The diagnoses were made by Italian primary neurological centres based upon anamnestic, clinical, evolutionary, neuroradiological and neurophysiological data.

Patients:

N1. Man, age 79 years old, first symptoms appeared four years ago.

UPDRS portion III before starting therapy 26 scores.

UPDRS portion III after two months of therapy 18 scores.

Improvement of motor symptoms by 30.8%

N2 Woman age 70 years old, first symptoms appeared three years ago.

UPDRS portion III before starting therapy 34 scores.

UPDRS portion III after two months of therapy 30 scores.

Improvement of motor symptoms by 11.8%

N3 Male age 56 years old, first symptoms appeared three years ago

UPDRS portion III before starting therapy 9 scores.

UPDRS portion III after two months of therapy 5 scores.

Improvement of motor symptoms by 55.5%

N4 age 75 years old, Woman, first symptoms appeared one year and a half ago.

UPDRS portion III before starting therapy 21 scores.

UPDRS portion III after two months of therapy 10 scores.

Improvement of motor symptoms by 47.6%

Multisystemic Atrophy (MSA) of Cerebellar Type (Type C)

We treated three patients affected by such pathology, a woman and two men with five vials intramuscularly per week of vitamin B1. A vial included 100 mg/ml of thiamine. The injection was performed every morning for the first five days of the week. The dose was determined empirically based upon the disease progression rapidity.

Moreover, low doses of all other vitamins of group b, including folic acid, 150 mg of magnesium citrate and 150 mg of magnesium malate, were associated to the treatment.

The patients were evaluated with SARA (Scale for Assessment and Rating of Ataxia), scale validated for all disorders of cerebellar type. The score of this scale ranges 0 (absence of symptoms) to 40 (maximum deficit). The treatment has lasted more than six months and the patients were evaluated before starting the treatment and two months after treatment beginning. They are currently under treatment by keeping the obtained results.

The diagnoses were made by Italian primary neurological centres based upon anamnestic, clinical, evolutionary, neuroradiological and neurophysiological data.

Patients:

N1. Man, age 73 years old, first symptoms appeared two years ago.

SARA scale before starting the therapy 7 scores.

SARA scale after two months of therapy 6 scores.

Improvement of motor symptoms by 14.3%

N2 Man, age 66 years old, first symptoms appeared two years ago.

SARA scale before starting the therapy 8 scores.

SARA scale after two months of therapy 3 scores.

Improvement of motor symptoms by 37.5%

N3 Woman age 56 years old, first symptoms appeared 5 years ago

SARA scale before starting the therapy 25 scores.

SARA scale after two months of therapy 21 scores.

Improvement of motor symptoms by 16.0%

Progressive Supranuclear Paralysis

We treated five patients affected by such pathology, two women and two men with five vials intramuscularly per week of vitamin B1. A vial included 100 mg/ml of thiamine. The injection was performed each morning for the first five days of the week. The dose was determined empirically based upon the disease progression rapidity. Furthermore, low doses of all other vitamins of the group b, including folic acid, 150 mg of magnesium citrate and 150 mg of magnesium malate, were associated to the treatment. The patients were evaluated with UPDRS (Unified Parkinson's Disease Rating Scale), scale validated for all Parkinson forms. Herein the scores of third portion (portion III) of this scale will be shown, illustrating the motor problems found by the examiner. The third portion ranges from 0 scores (no deficit) to 56 scores (maximum deficit), the treatment has lasted more than six months and the patients were evaluated before starting the treatment and two months after the treatment beginning. They are currently under treatment by keeping the obtained results.

The diagnoses were made by Italian primary neurological centres based upon anamnestic, clinical, evolutionary, neuroradiological and neurophysiological data.

Patients:

N1. Woman age 61 years old, first symptoms appeared one year and a half ago

UPDRS portion III before starting therapy 31 scores.

UPDRS portion III after two months of therapy 25 scores.

Improvement of motor symptoms by 20.3%

N2 Woman age 74 years old, first symptoms appeared nine years ago

UPDRS portion III before starting therapy 37 scores.

UPDRS portion III after two months of therapy 30 scores.

Improvement of motor symptoms by 19.9%

N3 Male age 64 years old, first symptoms appeared two years ago

UPDRS portion III before starting therapy 23 scores.

UPDRS portion III after two months of therapy 16 scores.

Improvement of motor symptoms by 32.5%

N4 age 75 years old, Woman, first symptoms appeared two years ago.

UPDRS portion III before starting therapy 28 scores.

UPDRS portion III after two months of therapy 22 scores.

Improvement of motor symptoms by 21.8%

N5 age 70 years old, Male. first symptoms appeared two years ago.

UPDRS portion III before starting therapy 28 scores.

UPDRS portion III after two months of therapy 20 scores.

Improvement of motor symptoms by 26.6%

Cortico-Basal Degeneration

We treated two patients affected by such pathology, two men with three vials intramuscularly per week of vitamin B1. One vial included 100 mg/ml of thiamine. The injection was performed in the three odd or even midweek days. The dose was determined empirically based upon the disease progression rapidity.

Furthermore low doses of all other vitamins of the group b, including folic acid, 150 mg of magnesium citrate and 150 mg of magnesium malate, were associated to the treatment.

The patients were evaluated with UPDRS (Unified Parkinson's Disease Rating Scale), scale validated for all Parkinson forms. Herein the scores of third portion (portion III) of this scale will be shown, illustrating the motor problems found by the examiner. The third portion ranges from 0 scores (no deficit) to 56 scores (maximum deficit), the treatment has lasted in a patient for about three years and in the other one for more than six months. Both of them were evaluated before starting the treatment and two months after the treatment beginning. They are currently under treatment by keeping the obtained results.

The diagnoses were made by Italian primary neurological centres based upon anamnestic, clinical, evolutionary, neuroradiological and neurophysiological data.

Patients:

N1. Man, age 78 years old, first symptoms appeared three years ago.

UPDRS portion III before starting therapy 26 scores.

UPDRS portion III after two months of therapy 15 scores.

Improvement of motor symptoms by 57.6% N2 Man age 74 years old, first symptoms appeared three years ago.

UPDRS portion III before starting therapy 35 scores.

UPDRS portion III after two months of therapy 31 scores.

Improvement of motor symptoms by 15.3%

Cluster Headache (Also Called Horton Headache)

The cluster headache is one of the most dangerous and intolerable headache types. The painful attacks have a periodic nature. The active phases, (called “cluster”, called in this way since the attacks are frequent and close and last 15 to 180 minutes each) last from weeks to months and alternate to long periods of spontaneous remission without pain. The localization is behind and around an eye. The causes are not known. It is believed that the hypothalamus, seat of our biological clock, could be involved.

Patient:

male, 41 years old, 94 kg, since he was 15 years old he has been suffering from typical episodes of cluster headache sensible only to treatment with subcutaneous Sumatriptan (selective agonist of serotonin). Several neuroradiological, neurophysiological and laboratory assessments produced no result. The situation has become chronic and in the last year he had 5-6 episodes of headache in 24 hours, each day treated each one with a subcutaneous vial of Sumatriptan.

The patient was evaluated with Cluster Headache Quality of Life (CHQ) scale before starting the treatment and every 15 days after starting the treatment which started in December 2016. The score of this scale ranges from 28 (absence of symptoms) to 140 (maximum deficit, maximum negative influence in daily life activities). The treatment has lasted for more than five months.

The patient started the therapy with 250 mg of thiamine orally, by increasing by 250 mg every three days. He had a progressive disappearing of the headache attacks and within about 8 days from the beginning, at the dose of 750 mg per day orally, the headache attacks wholly disappeared.

CHQ before treatment: 82 scores CHQ after 15 days of treatment: 28 scores

Then, a complete regression of the symptoms was obtained, which is maintained even today by continuing the treatment up to now. An attempt of interrupting the same led to the quick re-appearance of headache episodes therefore the patient started again the treatment and he did not interrupt it any more.

Migraine Headache

Migraine headache is the headache form characterized by a recurrent pain involving one only side of the head. The pulsating headache can last from four hours until three days and it is accompanied by nausea, vomiting, photophobia. The causes are poorly known. We studied two female patients starting from January 2017.

The patients were evaluated with Migraine Disability Assessment Test (MIDAS) evaluation scale before starting the treatment and every 30 days after starting the treatment, which started in January 2017. The score of this scale ranges from 0 (absence of symptoms) to 25 (maximum disability, maximum negative influence in the daily life activities). The treatment has lasted for more than 4 months.

Patients

N1: woman, 25 years old, weight 65 kg, weekly episodes of typical migraine headache, insensitive to each treatment, for about 5 years

The thiamine dose was 300 mg orally each morning.

MIDAS before treatment 19 scores. MIDAS after 30 days of treatment: 0 scores

N2. Woman, 70 years old. weight 75 kg, frequent episodes of typical migraine headache for about 40 years. In the last years such headache episodes were insensitive to each type of treatment and appeared twice a week.

The thiamine dose was 500 mg every morning orally.

MIDAS before treatment 20 scores. MIDAS after 30 days of treatment: 0 scores

The regression of the episodes was present already after 7 days of treatment and it is currently maintained by continuing the same treatment.

Determination of Vitamin B1 Concentration in Plasma

All patients before treatment showed widely normal values of vitamin B1 in plasma by suggesting that an administration of vitamin B1 was not required.

1. A method of treating motor symptoms of a pathology associated with Essential Tremor, Dystonia type DYT1, Huntington's Chorea, sporadic spinocerebellar ataxia, or atypical Parkinson diseases comprising administering a therapeutically effective amount of vitamin B1 to a subject in need thereof. Parkinsonisms, in particular multisystemic atrophy of Parkinson type and Cerebellar type, progressive supranuclear paralysis, Cortico-basal degeneration.

2. The method of claim 1, wherein the vitamin B1 is administered orally with a dosage ranging between 300 and 8000 mg per day.

3. The method of claim 1, wherein the vitamin B1 is administered orally with a dosage ranging between 2000 and 8000 mg per day.

4. The method of claim 1, wherein the vitamin B1 is administered intramuscularly with a dosage ranging between 50 mg and 100 mg once or several times a week.

5. The method of claim 1, wherein the vitamin B1 is administered intramuscularly with a dosage ranging between 50 mg and 100 mg, preferably 100 mg at least once a day.

6. A method of treating motor symptoms of a pathology associated with Essential Tremor, Dystonia type DYT1, Huntington's Chorea, Sporadic spinocerebellar ataxia, or Atypical Parkinson diseases comprising administering a therapeutically effective amount of a pharmaceutical composition comprising vitamin B1 and one or more pharmacologically acceptable excipients to a subject in need thereof.

7. The method of claim 6, wherein the pharmaceutical composition is in the form of granules, powders, solutions, capsules, tablets, tablets, lyophilized tablets, pills for oral administration, solutions for intramuscular administration.

8. The method of claim 6, wherein the vitamin B1 is in an amount ranging from 250 and 8000 mg, in particular between 250 and 1000 mg, per dosage unit.

9. The method of claim 7, wherein the composition is administered orally with a dosage ranging between 2000 and 8000 mg per day.

10. The method of claim 6, wherein the composition is administered intramuscularly with a unit dosage of about 100 mg twice a week or once a day.

11. (canceled)

12. A method of treating cluster headache and/or migraine headache comprising administering a therapeutically effective amount of vitamin B1 to a subject in need thereof.

13. The method of claim 12, wherein the vitamin B1 is administered intramuscularly with a dosage ranging between 25 and 100 mg at least once a week.

14. The method of claim 12, wherein the vitamin B1 is administered orally with a dosage ranging between 300 and 2000 mg at least once a day.

15. The method of claim 12, wherein said migraine headache is chronic or intermittent.

16. A method of treating cluster headache and/or migraine headache comprising administering a therapeutically effective amount of an oral pharmaceutical composition comprising vitamin B1 in an amount ranging between 300 and 500 mg per dosage unit and one or more pharmacologically acceptable excipients to a subject in need thereof.

17. A method of treating cluster headache and/or migraine headache comprising administering a therapeutically effective amount of an intramuscular pharmaceutical composition comprising vitamin B1 in an amount ranging from 25 and 100 mg per dosage unit and one or more pharmacologically acceptable excipients to a subject in need thereof.

18. The method of claim 1, wherein the atypical Parkinson diseases are multisystemic atrophy of Parkinson type and Cerebellar type, progressive supranuclear paralysis, or Cortico-basal degeneration.

19. The method of claim 6, wherein the atypical Parkinson diseases are multisystemic atrophy of Parkinson type and Cerebellar type, progressive supranuclear paralysis, or Cortico-basal degeneration.

20. The method of claim 13, wherein the vitamin B1 is administered at dosage ranging between 25 and 50 mg.

21. The method of claim 17, wherein the vitamin B1 is administered at a dosage unit between 25 and 50 mg.
